(19) United States
(12) Patent Application Publication
(10) Pub. No.: US 2024/0065882 A1
(43) Pub. Date: Feb. 29, 2024

(54) SMART THERMOELECTRIC BRAIN HYPOTHERMIA THERMOHIPOTHERM AND BRAIN THERMOGRAPHY DEVICE

(71) Applicant: TES THERMOELECTRIC LTD, Ankara (TR)

(72) Inventor: Rasit AHISKA, Ankara (TR)

(73) Assignee: TES THERMOELECTRIC LTD, Ankara (TR)

(21) Appl. No.: 18/270,552

(22) PCT Filed: Nov. 26, 2021
(86) PCT No.: PCT/TR2021/051301

§ 371 Date: Jun. 30, 2023

(30) Foreign Application Priority Data
Dec. 31, 2020 (TR) 2020/22699

(51) Int. Cl. A61F 7/00

(52) U.S. Cl. CPC A61F 7/007; A61F 2007/0008; A61F 2007/0056; A61F 2007/0075; A61F 2007/0079; A61F 2007/008; A61F 2007/0093

(57) ABSTRACT

A smart thermoelectric brain hypothermia thermohipoetherm and brain thermography device cools or heats the human brain from the outside quickly with temperature control by converting the electrical energy received from the vehicle battery or the electrical network directly into cold or heat through a flexible thermoelectric helmet consisting of N and P-type semiconductors without using any intermediates. The device is equipped with smart software, easily portable, and flexible, and has a fluid circulation system to perform brain thermography to the patient's brain from the outside in a short period of time and without harming the patient and the environment, such as the harmful effects from radiation and magnetic fields. The device can perform a treatment that can keep the brain at a temperature of 28° C.-30° C. At the same time, this device can diagnose all kinds of diseases by taking brain thermography.

### TECHNICAL FIELD

The invention can cool or heat the human brain from the outside quickly with temperature control by converting the electrical energy received from the vehicle battery or the electrical network directly into cold or heat through a flexible thermoelectric helmet consisting of N and N P-type semiconductors without using any intermediates. The invention is the new “Smart Thermoelectric Brain Hypothermia and Brain Thermography Device,” equipped with smart software, easily portable, flexible, with a fluid circulation system. It can perform brain thermography without harming the patient and the environment. The first area where the invention is concerned technically falls into the field of external brain cooler and/or warming machine, brain hypothermia medical device engineering.

### BACKGROUND

External brain cooler and/or warming machines or brain hypothermia medical devices are electrically powered machines. These machines are divided into two groups as machines that can use intermediate or direct cooling. External brain cooler and/or heating machines or brain hypothermia medical devices that operate using intermediates are coolers that perform cooling with externally cooled water or liquid such as antifreeze. Machines that can directly cool or heat without the use of intermediates are thermoelectric devices. Another field related to the invention is the tomography machine, which is technically one of the brain neuro-imaging techniques, and brain thermography medical device engineering field. Brain imaging and tomography medical devices are electrically powered machines. These devices do not cure, only imaging and are used for diagnostic purposes. The working principles of the current brain tomography device, which is not portable and quite expensive (GE Brand Brain Tomography Device with a price of at least $250,000), is to deliver radioactive material to the patient and to take images of the brain or any other organ or any part of the body in a high magnetic field. Therefore, a patient undergoing tomography is exposed to very harmful high radiation and a strong magnetic field. The radiation exposure during tomography is equal to the radiation exposure in 8 months of daily life. Brain tomography has high risks in pregnancy and newborns. In many diseases, especially in psychiatric cases, brain imaging methods and the brain mapping technique that forms the basis of these methods have become increasingly important in recent years. Techniques used for brain mapping (CT scan technique, PET, fMRI, EEG techniques come to the fore. CT Scan (Neuroscience) technique is all based on the use of X-rays. If a narrow X-ray beam passes through the same object from multiple angles, it is possible to use computational techniques to create a visual image of the brain. However, prolonged or repeated exposure of the patient to radiation causes tissue damage. In addition, only structural information about the brain can be collected. This is great for identifying problematic brain tissue, but it gives us little information about how the brain functions during cognition. Again, the heat mapping technique of the brain that exposes the patient to radiation and the thermograph device applied to it constitute the closest known state of the invention.

While the heat map of the brain gives the heat distribution of the brain in the first place, it is important in terms of the working properties of the brain and the detection of the areas with bleeding and overheating. Here, 3D brain temperature mapping is a highly enlightening evaluation method. In thermal brain mapping (brain thermography), brain temperature is highly dependent on the metabolic activities of tissues in the brain and blood flow in the brain. It was observed that the blood flow rate caused a regional temperature increase.

Brain thermography is one of the advanced technological methods currently used in “diagnosis, enhancement of diagnosis, measurement, and evaluation of treatment results.” In many psychiatric and other diseases, pre-and post-treatment 3D images are compared, and treatment results can be quantitatively evaluated. Thus, it provides the right decisions to choose the appropriate treatment for various diseases and whether to continue or terminate it. Compared to traditional neuroimaging techniques such as fMRI and fNIRS, the thermography method is a more economical and portable. In addition, using mapping techniques, the heat map of the brain can be derived using image processing techniques.

In the prior art, inventions involving thermoelectric modules in Turkey are as follows:
1. 1993/26708 Thermoelectric device to influence the organism with temperature
2. 1995/01333 An ecological thermoelectric cooling system.
3. 1999/00284 Peltier microprocessor cooler
4. 2006/02583 Liquid cooler
5. 2006/05431 Commercial countertop cooler.
6. 2007/03485 Thermoelectric cooling/heating device.
7. 2008/01140 Temperature control system with Peltier.
8. 2008/09884 Quick ice-making units.
9. 2012/15422 Peltier portable distiller
10. 2013/15041 Device for regulating the temperature of a physiological fluid.
11. 2014/14588 Desktop thermoelectric ice maker.
12. 2016/08411 Thermoelectric refrigerated supermarket type display section.
13. 2016/06918 Portable temperature regulator for medicines.
14. 2017/00051 Systems and methods of a thermoelectric heat exchange system.
15. 2017/11700 Thermoelectric vehicle liquid cooler
16. 2019/16734 Hyperthermia and Hypothermia Thermoelectric Incubator System
17. 2014/03283 Computer con rolled cold and hot therapy device with the thermoelectric module

Other inventions are as follows:
1. U.S. Pat. No. 10,716,703 Devices and methods for controlling patient temperature
2. U.S. Pat. No. 10,716,703 Devices and methods for controlling patient temperature
3. U.S. Pat. No. 9,326,890 Devices and methods for controlling patient temperature
4. U.S. Pat. No. 9,301,871 Devices and methods for controlling patient temperature
5. U.S. Pat. No. 9,320,644 Non-invasive systems, devices, and methods for selective brain cooling
6. U.S. Pat. No. 8,012,116 Device to reduce brain edema by surface dialysis and cooling
7. U.S. Pat. No. 7,228,171 Signal analysis, heat flow management, and stimulation techniques to treat medical disorders
8. U.S. Pat. No. 6,923,826 Neurosurgical device for thermal therapy
9. U.S. Pat. No. 6,899,726 Expandable device for thermal therapy including spiral element
10. U.S. Pat. No. 6,743,200 Expandable device for thermal therapy
11. U.S. Pat. No. 6,660,026 Multi-tipped cooling probe
12. U.S. Pat. No. 6,652,566 Neurosurgical device for thermal therapy including spiral element
13. U.S. Pat. No. 6,648,907 Neurosurgical device for thermal therapy
14. US2013207617 Thermal Management System For A Battery System
15. US2002073716 Thermoelectric cooler temperature control
16. US2004251539 Thermoelectric cooler array
17. US2004069339 Thermoelectric cooler having first and second TEC elements with differing physical parameters
18. US2004031272 Thermoelectric cooler controller

### BRIEF DESCRIPTION AND OBJECTS OF THE INVENTION

Smart Thermoelectric Brain Hypothermia Thermohypotherm and Brain Thermography Device is designed in the form of a helmet. Brain hemorrhages, hypersensitive brain parts can be detected, and a continuous and uninterrupted flow of data can be provided about the course of the disease. Brain imaging is usually performed in clinical settings using electrodes placed on the brain. Dozens of electrodes are used for this. This data collection technique, which must be connected externally, is a technique that is difficult to apply. Since the invention consists of 120 flexible thermoelectric thermoelements, when the helmet is worn on the patient, it provides not only the cooling or heating function at different points but also the amount of heat released from these points and the measurement of the temperatures in the brain depth we want. The new Smart Thermoelectric Brain Hypothermia Thermohypotherm and Brain Thermography Device can diagnose and map the brain's temperature with techniques such as 3D brain heat mapping diagnosis and hypothermia.

The invention eliminates the disadvantages (radiation, strong magnetic field, radioactive material, high price, portable and non-portable) existing brain hypothermia, brain tomography, and thermography devices and systems, as well as collecting data by simply placing the helmet on the patient's head without the need for any electrode. The new Smart Thermoelectric Brain Hypothermia Thermohypotherm and Brain Thermography Device is practical, inexpensive, portable, harmless to patients and the environment, and it can receive uninterrupted information in any environment regardless of clinical conditions due to its easy application.

The benefits of this invention are:
1. The invention can be an alternative to brain hypothermia and brain thermography devices used in medicine and can be used on various patients, especially on Covid-19 patients. It can perform treatment and diagnosis without causing any harm to the patient and also has an intelligent measurement, recording, and control system. The device is a new invention with a portable and inexpensive construction. It also provides great ease of use to doctors. This will continuously increase the area of use and the number of uses of the invention. In addition, since the product is related to human health, it will serve all humanity. Understanding this will increase the importance of the use of the invention.
2. Due to its features, the invention will greatly contribute to medical science and especially brain hypothermia research. Since basic scientific research and R&D studies will be carried out in an easier, more practical, cheaper, and more reliable manner with the device, the development of technologies and the production of new medical devices in areas such as medicine, hypothermia, thermoelectricity, medical electronics, and software will be accelerated. As a result, with the establishment of Hypothermia R&D centers in the world (currently there are only two centers in the USA and Europe and the feature used in these centers are very primitive brain hypothermia methods and systems), their geography will expand, and their number will increase rapidly, especially in Turkey.
3. The smart thermoelectric brain hypothermia thermohypotherm and thermography device is original and high added value in Turkey and the world, which will greatly contribute to the country's export and country's economy and prestige. The invention is a product with a high probability that it will be required in all patient transport vehicles, especially in ambulances.
4. Since the invention does not use gas and ice like all thermoelectric devices, it will significantly contribute to public health, protection of the environment, and clean water savings.
5. Since the smart thermoelectric brain hypothermia thermohypotherm and thermography device can also work with a battery, the invention can easily be used in ambulances and all kinds of civil and military transport vehicles.
6. Since the invention is portable, it can be easily used anywhere and in any environment.
7. When the sales price of the invention is at least 10 times cheaper than the cheapest of the current brain hypothermia and brain tomography devices, it will provide great convenience to the consumer.
8. All production of the invention will be domestic. The production of these devices in Turkey for the first time in the world will contribute to the solution of the employment problem of the country by creating a new flexible thermoelement and flexible-based thermoelectric module industry in the country. It will also eliminate Turkey's dependence on medical technologies such as high thermoelectric and brain thermography.

### BRIEF DESCRIPTION OF THE DRAWINGS

The “Smart thermoelectric brain hypothermia thermohypotherm and thermography device” produced to achieve the purpose of the invention is shown in the attached FIGURE.

FIG. 1 illustrates the architecture of the invented product. FIG. 1—is the block diagram of the smart thermoelectric brain hypothermia thermohypotherm and thermography device.

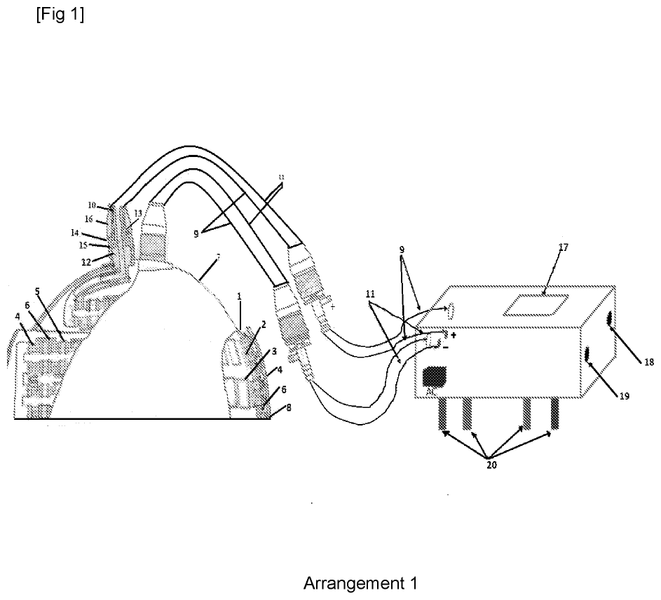

### DESCRIPTION OF THE REFERENCES

The parts in the FIGURE are as follows with their sequence numbers.
1) Flexible Antibacterial Soft Bottom Plate
2) Copper Sheet of Flexible Thermoelement
3) Slot
4) Copper Tube Providing Liquid Circulation-Cold Collector
5) Flexible Silicone Hose Providing Thermoelements to Connect Without Hydraulically Leaking
6) Flexible Multiple Copper Wire for Electrically Connecting Thermoelements to Each Other
7) Flexible Soft Top Sheet
8) Filler that Fills the Space Between the Thermoelements that are between the Top and Bottom Plates
9) Flexible Combined Hose Consisting of Water Pipes and Electric Wires
10) Combined Hose Fitting
11) Socketed Electrical Connection Cables
12) Metal Bracket Fixed on The Helmet
13) Metal Connection Part Fixed to the End of the Hose
14) The Rough Part of the Metal Connection Part Fixed on the Helmet
15) Connection Tab
16) Rough Part of the Metal Fitting Fixing to the End of the Hose
17) Touch Control Panel of the Power Control Unit (PCU)
18) ACU On/Off Button
19) ACU Led Indicator
20) ACU Wheels

### DETAILED DESCRIPTION

Smart Thermoelectric Brain Hypothermia Thermohypotherm and Thermography Device consists of 4 main parts: a thermoelectric cooler (helmet); Electronic circuits (DC-DC converter) that enable the flexible thermoelectric helmet (module) to use the correct (DC) electrical voltage and/or alternative (AC) voltage (220/380 ACV) by converting it to DC voltage; AC-DC mains connected and/or disconnected power supply; preferably a battery; a power supply with Switch Mode Power Supply (SMPS) consisting of a charging unit; An electronic control unit with an LED indicator that makes the thermohypotherm device smart, can show, measure and record the temperature, flow rate and the amount of heat released from the brain; and a recirculation unit that works with a liquid such as water or antifreeze that keeps the surface temperature constant by removing the heat of the heated surface of the flexible thermoelectric helmet (module).

The device is manufactured as a single piece of a thermoelectric cooler helmet, a surface-cold collector consisting of the cooling surfaces of the sheets of flexible thermoelements, a hot collector consisting of pipes of flexible thermoelements, electrical connection cables, and flexible silicone hoses that provide uninterrupted fluid circulation through the pipes. Thermohypotherm and Thermography device has a power supply, control and circulation units, AC220V input on the back panel, DC5-12V and DC0-50 A electricity and water input-output, on-off button on the front panel, LEDs, and touch-sensitive adjustment control and display panel on the top.

The device is manufactured as a single piece and has a portable power-control unit connected to a computer. The thermoelectric cooler (TE helmet) and GKU are hydraulically connected with hoses using plug-in fittings. Thus, the invention is a Smart Thermoelectric Brain Hypothermia Thermohypotherm and Thermography Device made of connection cables and hoses selected according to medical standards and especially flexible combined hoses produced according to medical standards, and GKU equipped with TE helmet and smart software.

The scientific basis, theoretical and experimental researches of the methods, and scientific findings of the solutions of the technical problems solved by the invention have been made. For this purpose, a device was manufactured, clinical studies were conducted in a university hospital with the permission of the ethics committee, and studies are ongoing. The Smart Thermoelectric Brain Hypothermia Thermohypotherm and Brain Thermography Device is used as a treatment (Brain Thermohypotherm) and diagnosis (Brain Thermography) device for brain diseases, anesthesia, cardiology, and other real patients, especially Covid-19 patients. At the same time, scientific basis, theoretical and experimental research are carried out here. The theoretical and experimental scientific findings obtained from these studies form the basis of the solutions of technical problems solved by the invention. The technical problems solved in the invention consist of two groups. The first group consists of the working principle of the invention as a treatment (Brain Thermohypotherm) device and the technical problems it solves. As a result of scientific research, the most basic scientific finding accepted by medical science and witnessed by people almost every day is that whatever the disease is, the brain of a person or a living being is more or less important, insignificant, temporarily or temporarily permanently sick is affected. The human brain is a 1.5-kilogram miracle. It uses 25% of the oxygen and most of the glucose that enters our body. But the weight it occupies in the body is 2%. 2% brain consumes 25% energy. The brain, in which thoughts, emotions, and behaviors are managed, is a special organ. According to scientific findings, the brain temperature of a healthy person is always higher than the body's and is around 37.5° C. The thermal power released by an adult healthy human body is 172 W. 70% (120 W) of this thermal power comes out from the brain and the human skull. Accordingly, this thermal power must be discharged to cool a person's brain. Therefore, to cool the brain from the outside, the cooling capacity of the device that will cool the skull should be at least 120 W. The first and only invention, which has a cooling capacity of at least 120 W, designed, produced, and solved a technical problem based on this scientific finding, is the Smart Thermoelectric Brain Hypothermia Thermohypotherm and Thermography Device. None of the current methods and devices used for brain hypothermia have a cooling capacity of 120 W, and none can solve this basic technical problem.

Hyperthermia occurs when the human body temperature rises above the normal level. Hypothermia occurs when the body temperature drops below normal. In both cases, the brain must be protected. In addition, in cases of trauma and open-heart surgeries, if the heart pauses for any reason, cell death begins in the brain in a short time. To prevent this; The brain needs to be cooled quickly. Again, according to scientific findings, the most important factors affecting the brain are severe pain, high fever, and hypoxia (lack of oxygen), especially for Covid-19 patients, brain diseases, anesthesia, cardiology. In all cases, the patients' lives can be saved when the brain is cooled to 28° C.-30° C. in a rapid and controlled manner. Accordingly, the only solution to protect these patients' brains or save their lives is brain hypothermia. In other words, it is cooling the brain and keeping it cold (28° C.-30° C.). In addition, it is known from Ibn-Sina that hypothermia, especially brain hypothermia, is a treatment method and a method of anesthesia. There are thousands of articles in the literature on this. However, as in the world, the hypothermia method is used very little in Turkey. The only reason for this is that the current hypothermia devices, especially the existing brain cooling devices (currently used in the world in general hypothermia, a bath filled with cold water or pad with cold water in it, and for brain hypothermia, helmets or ice packs with cold water are used) have less than 120 W cooling capacity, and cooling rates are not at least 0.5° C./min, and they are not practical and useful. Nowadays, brain hypothermia is performed by immersing the whole body in cold water. With this method, it is not possible to quickly reduce the brain temperature to the desired temperature. Because the body must be kept in cold water for a long time in order to lower the brain to the required temperature, in this case, the heart is stressed, and dangerous complications occur. For this reason, it is very important to cool the brain with the local cerebral (external) hypothermia method. On the other hand, in local hypothermia performed by putting ice on the head, the desired cooling rate and temperature drop cannot be achieved. In addition, the use of ice causes environmental pollution and waste of clean water. For this reason, research is carried out on local cerebral hypothermia techniques and methods, which do not cause any complications in the world and are very practical. The technology that will solve the main problems here is Smart Thermoelectric Brain Hypothermia Thermohypotherm and Brain Thermography Device (STEBHTBTD). The device is a treatment device with the necessary hardware and software to cool the brain up to 30° C. in a fast and controlled manner in brain diseases, anesthesia, cardiology, and other cases, especially Covid-19 patients exposed to severe pain, high fever, and hypoxia.

After sudden cardiac arrest, there is a decrease in blood pressure, and the amount of blood flowing to the brain decreases. Cellular damage occurs due to cerebral circulation disruption. Brain cells have a delicate structure; cellular damage starts quickly and takes 3-5 minutes. It can progress to an irreversible level in a short time. Effective cardiac massage is required to minimize this and similar damages, and the intracranial temperature (fever) should be lowered to reduce the cellular oxygen consumption of the brain and reduce the area of damage. Nowadays, ice packs are placed around the head directly to reduce the intracranial temperature, or the body is completely wrapped with ice packs, or a pad with cold water or a helmet with cold water (such as Olympic Cool-Cap and Surface Cooling/Arctic Sun 5000) or ice packs are used. Although such methods are not effective, they are not practical since the cooling capacities of the devices are way less than 120 W and 0.5° C./min. The Smart Thermoelectric Brain Hypothermia Thermohypotherm and Thermography Device, which solves these basic problems, is a medical device that can effectively control cooling the brain and bring it back to normal temperature by heating, when necessary, that is, it can perform two functions such as cooling and heating with a single device and does not harm the patient. In addition, a method with successful results in experimental animal studies and which does not harm the human body was developed with this device. A Thermoelectric Thermohypotherm cooling system can be used to achieve either superficial or deep craniocerebral hypothermia.

The outstanding parameters of a brain cooler are its cooling capacity and speed. The cooling capacity of existing brain coolers such as Surface Cooling/Artic Sun 5000 and Olympic Cool-Cap helmet with cold water circulation is below 120 W, and the cooling rate is well below 0.5° C./min. After hypothermia, the brain needs to be brought to normal temperature, and these devices cannot heat the brain in a controlled manner (at a rate of 0.5° C./min) (with this device, the patient is abandoned after hypothermia and the patient's brain warming is done naturally, uncontrolled and at low speed). Brain hypothermia with these devices makes the treatment inadequate and ineffective and narrows the usage area of the brain hypothermia method and reduces the confidence in this method, which is very effective and harmless.

The second group consists of the working principle of the invention as a diagnostic (Brain Thermography) device and the technical problems it solves. The invention can only measure and diagnose the temperature and the amount of heat in the brain (brain at different depths) without causing any harm to the patient. At the same time, it treats local hypothermia by direct contact with the skull without using any intermediates, and it is portable, easily transportable, and very cheap.

Smart Thermoelectric Brain Hypothermia invention was designed and produced as a brain thermography device according to clinical research on real patients and the examination results of flexible thermoelements used in the production of helmets and the new calculation formulas obtained with Thermohypothermia and Brain Thermography device. The cooling capacity of a thermoelectric helmet or a thermoelement in Watt is Q_{C}, and the temperature of the cold surface in Kelvin is T_{C}.

$${Q}_{C} = {V}_{max} \cdot I - \frac{0 , 5 \cdot {I}^{2} \cdot ( {V}_{max} - {E}_{max} )}{{I}_{max}} - ( I + \frac{0 , 5 \cdot ( {V}_{max} - {E}_{max} ) \cdot {I}_{max}}{{E}_{max}} ) \cdot E ve {T}_{C} = {T}_{H} ( 1 - \frac{E}{{V}_{max}} ) \qquad [ Math . 1 ]$$

Here, the current drawn by the working helmet (I), the voltage falling at its ends (V), the thermoemic (E) it produces, and the temperature on its heating and cooling surface (T_{H} or T_{C}) are the experimental parameters Vmax, Iₘₐₓ and Eₘₐₓ. In addition, Q_{C} refers to the thermal power released from the brain, and TC refers to the skull and brain surface temperature. According to these formulas or this new theory (method), the brain temperature and the amount of heat coming from the brain (with the same device at the same time) are easily measured with a helmet. These basic scientific findings have enabled the identification and solution of the technical problems required to reduce the cooling capacity of the device to at least 120 W cooling rate 0.5° C./min and reduce the brain from 40° C. to 30° C. in 5 minutes at most.

Various cases have been treated with the device for a while. While treating these patients, necessary findings were obtained, and scientific researches were also carried out. Here, body temperature measured in degrees Celsius is (T_{b}), the intra-ear temperature is (Tₑ), and 1 cm brain depth temperature is (T₁), 2 cm depth temperature is (T₂), 3 cm depth temperature is (T₃), 4 cm depth temperature is (T₄), and 5 cm depth temperature is (T₅). The correlation values between them were found as 0.944, 0.958, 0.967, 0.975, 0.976, and 0.980, respectively. These results were accepted as justification, and for the first time in the world, linear functions (formulas) were obtained between body temperature and other temperatures.

Tₑ=−70.6+2.74*T_{b}

T₁=−220.9+6.49*T_{b}

T₂=−185.6+5.62*T_{b}

T₃=−174.3+5.37*T_{b}

T₄=−164.1+5.12*T_{b}

T₅=−158.2+5*T_{b} [Math. 2]

TABLE 1
Cooling Feature of the Brain Thermography Device

| Time, min | Ear, ° C. | Body, ° C. | 5 cm, ° C. | 4 cm, ° C. | 3 cm, ° C. | 2 cm, ° C. | 1 cm, ° C. |
|---|---|---|---|---|---|---|---|
| 0 | 38.31 | 39.51 | 39.51 | 38.79 | 38.55 | 37.35 | 36.87 |
| 15 | 37.11 | 39.27 | 39.03 | 37.83 | 37.55 | 35.91 | 35.43 |
| 30 | 36.39 | 39.27 | 37.83 | 36.63 | 36.39 | 34.71 | 33.52 |
| 45 | 35.91 | 39.03 | 36.63 | 35.43 | 34.95 | 33.52 | 31.84 |
| 60 | 35.43 | 38.79 | 35.43 | 34.47 | 33.76 | 31.84 | 30.16 |
| 75 | 34.71 | 38.79 | 34.47 | 33.04 | 32.56 | 30.64 | 28.96 |
| 90 | 34.23 | 38.31 | 32.08 | 31.36 | 30.64 | 28.96 | 26.56 |
| 105 | 33.76 | 37.83 | 31.12 | 30.16 | 29.44 | 28 | 25.61 |
| 120 | 33.04 | 37.59 | 29.92 | 28.96 | 28.24 | 26.56 | 24.41 |

The average of the absolute error margins between the temperature values calculated with these formulas and the values in the directly measured figure are respectively 0.5° C.; 1.1° C.; 0.81° C.; 0.66° C.; 0.61° C.; 0.54° C. Thus, the device, a completely domestic and national product, is a temperature-controlled smart brain cooler and treatment device and a smart new device that can measure and record temperatures at various depths quickly and precisely without interfering with the human brain. These basic scientific findings have led to a device that can measure and record the temperatures of the brain at various depths and the amount of heat released quickly and very precisely without interfering with the human brain. Likewise, it has provided the identification and solution of technical problems required for an easily portable and inexpensive thermography device that can convert to 3D images, diagnose without causing any harm to the patient.

According to the basic research in medicine and the scientific findings obtained, when the human body is considered a whole, the damage or change resulting from a trauma or a disease in any part of the body directly affects the human brain. As a result, the metabolism itself, the nature and speed of metabolism in the brain changes. These changes alter the temperature of the brain and the distribution of temperature in the brain. Therefore, by measuring the amount of heat released from the brain, the temperature of the brain, and by determining the temperature distribution in the brain in terms of both surface and depth, we can diagnose cases that cause changes not only in the brain but in any part of the body. The only technology and product that can do this are Smart Thermoelectric Brain Hypothermia Thermohypotherm and Brain Thermography Device.

On the other hand, existing brain tomography devices do not treat only imaging and are used for diagnostic purposes. A patient who has to use these non-portable and very expensive devices is exposed to very harmful high radiation and severe magnetic field.

The invention uses scientific findings obtained by conducting theoretical and experimental research on these basic brain hypothermia and tomography problems. The newly developed flexible thermoelement, flexible helmet, applying new temperature and new temperature measurement technologies makes the cooler in the shape of a human skull. Accepting that the first 2 hours, which is a scientific finding, is critical for recovering a damaged brain, it cools the brain locally from 40° C. to 30° C. in a maximum of 5 minutes with a cooling capacity of at least 120 W and a cooling rate of 0.5° C./min. It diagnoses all kinds of diseases by using electrical energy with maximum efficiency and by applying cooling or heating rapidly, with smart control, hypothermia, and at the same time without causing any harm to the patient.

Smart Thermoelectric Brain Hypothermia Thermohypotherm and Thermography Device consists of a thermoelectric helmet (TEH) and a power control unit (PCU) equipped with smart software and special flexible combined hoses consisting of water pipes and electric wires (9) with two sockets that provide the electricity and water connection between TEH and PCU, which tin connect (or separate) them to each other. The smart thermoelectric brain hypothermia thermohypotherm and thermography device consists of the following: Thermoelectric flexible helmet, flexible thermoelements made of a soft, insulating, anti-bacterial material like leather and placed in 120 slots on it, bottom plate, water collectors that provide fluid circulation through copper pipes of thermoelements, connect them to each other. Flexible silicone hoses that allow hydraulic connection, flexible multi-copper wire that allows thermoelements to be electrically connected to each other, top sheet made of a flexible soft, insulating, antibacterial material like leather that covers all these parts, flexible filler filling the spaces between the thermoelements between the lower and upper sheets, flexible thermoelectric module helmet that can take the shape of a human skull, a metal connector made as a hemisphere and fixed on the thermoelectric flexible helmet, a flexible combo consisting of plug-in electrical connection cables, water pipe and electrical wires Metal fitting and combined hose fitting fixed to the end of the hose, the rough part of the fitting fixed to the helmet, the rough part of the fitting fixed to the combined hose (flexible combined hose, FCH) end, closed water pump to remove the heat released from the heated surface of the thermoelements inside a casing, radiator-fan circulation system, DC SMPS power supply is placed and smart software equipped to make the helmet more efficient and economical, touch control panel, on-off button placed on the front panel, LED indicator, connection to a power control unit (PCU) consisting of four wheels, fixed on the rear panel of the case. TEH+FCH+PCU systems that are easily connected and separated from each other, created by easily connecting with parts.

All these special parts that make up the invention have been calculated, designed, and produced based on the information and findings obtained from basic scientific, theoretical, and experimental research. The invention, the main technical problems solved by the invention, especially in cases such as brain trauma, heart attack, and Covid-19, in all cases without exception, the brain of the sick person in a short time of 5 minutes with a cooling capacity of at least 120 W and cooling at 0.5° C./min. It cools locally in a controlled manner with its speed. It keeps the brain temperature at 28° C.-30° C. for the required time and ensures that the brain is heated to a normal temperature with a controlled speed of 0.5° C./min after hypothermia. The device is designed to perform the brain thermography procedure to diagnose the patient cheaply and practically without any harm.

For this, the basic technical problems to be solved by each of the parts, such as flexible thermoelectric helmet (headgear), PCU, and flexible combined hose that make up the device, were determined and solved. In this regard, the things realized in the invention are as follows.

The flexible thermoelectric helmet, which is firmly attached to an adult's head (can be designed in various shapes for children), consists of flexible thermoelements connected by flexible silicone hoses providing thermoelements to connect without hydraulically leaking (5), in series and thermally parallel to each other, flexible anti-bacterial soft bottom plate (1) and top plate, and flexible filler that fills the space between the thermoelements that are between the top and bottom plates (8). Thus, when the skull of a sick person with a helmet is cooled from the outside, since the helmet grips the patient's skull tightly from all sides, all the cooling plate surfaces of the flexible thermoelements directly contact the skull, and the helmet cools or heats the brain rapidly (at least 0.5° C./min.). For this, the inner surface dimensions of the helmet were produced in accordance with the surface of the skull. In addition, the size, power, and number of thermoelements are designed to solve the second problem that needs to be solved by throwing 120 W of thermal power released from the human brain. The invention can cool the brain temperature of a person who is suffering from any disease or who is healthy, up to a temperature of 28° C.-30° C. with a cooling or heating rate of at least 0.5° C./min in a maximum of 5 minutes, or it can warm a cooled brain back to its normal temperature of 37.5° C. (according to research, the brain temperature of a healthy person is always higher than the body's, and it is around 37.5° C.).

The thin flexible, high heat conductive and electrically insulating material covering the inner surface of the thermoelectric helmet with a thickness of 1.5 mm is made in accordance with health standards, easily cleaned, with antibacterial surfaces. The invention scientifically revealed for the first time the ability to measure the temperature and amount of heat they have separately and as a single flexible thermoelectric module of the helmet with the flexible thermoelements that make up the thermoelectric helmet, not only to cause hypothermia, but also to determine the temperatures of the brain at various depths and the amount of heat released from various regions. By measuring and recording the temperature and heat maps of the brain, it can diagnose all kinds of diseases.

Flexible thermoelectric helmet-module and flexible thermoelement were calculated, designed, and produced based on the information and findings obtained due to basic scientific theoretical and experimental research. The DC power drawn by the flexible thermoelectric helmet module is 12V×40 A=480 W, the cooling capacity is 240 W. The module is specially made from 120 flexible thermoelectric semiconductors, 120 pieces of N, and 120 pieces of P-type thermoelectric semiconductors with a diameter of 8 mm and 5 mm. Flexible thermoelement is produced by soldering multiple copper wires to one side of thermoelectric semiconductors produced as 1 piece of N and 1 P-type cylindrical or straight parallelepiped a copper tube with a flat side on the other side with a special solder. Thus, the thermoelectric cooler (helmet) is manufactured as a single piece (thermoelectric helmet/helmet) from flexible silicone hoses that provide continuous fluid circulation from electrical connection cables and pipes, the surface-cold collector consisting of the cooling surfaces of the sheets of flexible thermoelements, the hot collector consisting of pipes of flexible thermoelements. The invention is a new smart thermoelectric brain hypothermia thermohypotherm and thermography device that consists of flexible thermoelements and has a flexible helmet module placed on a flexible antibacterial soft bottom plate (1).

The closed liquid recirculating heat transfer system, which forms the special power-control unit (PCU) of the Thermohypotherm and Thermography device, has been calculated, designed and produced based on the information and findings obtained as a result of basic scientific theoretical and experimental researches. In the invention, a special closed liquid circulating heat transfer system consisting of circulating connection hoses, circulating DC or AC pump, circulating radiator and circulating radiator fan was made to cool the heated surface of the flexible thermoelement and special flexible thermoelectric helmet—thermoelectric module. According to calculations, a thermal power of at least 240 W should be discharged from the heated surface of the helmet for 120 flexible thermoelement thermoelectric helmet-modules to cool the patient's brain temperature by at least 0.5° C./min cooling rate in 5 minutes by 28° C.-30° C. In the invention, flexible thermoelement technology combined with flexible thermoelectric helmet technology for the first time in the world. For this, a heat transfer system with a closed liquid circulation fan that can discharge at least 240 W of thermal power has been produced. The invention is a new smart thermoelectric brain hypothermia thermohypotherm and thermography device with a closed liquid circulating fan heat transfer system capable of delivering at least 240 W of thermal power.

The special DC SMPS power and temperature control unit, which creates the special power-control unit (PCU) of the Thermohypotherm and Thermography device and enables the thermoelectric helmet to work with the vehicle battery or electrical network, has been calculated, designed, and produced based on the information and findings obtained as a result of basic scientific theoretical and experimental research. The DC power and temperature control unit, which will be used in the invention and connected to the thermoelectric helmet with electrical connection cables, has been provided with high efficiency and high reliability according to medical standards. DC power, temperature measurement, recording, and control unit are produced in accordance with the required EN norms, 220 VAC powered, output at least DC 12V×50 A and 4 KV DC galvanically isolated or at least 12 DCV×60 A vehicle battery powered. The unit is a special DC power, temperature measurement, recording, and control unit that will provide the required output parameters to the flexible thermoelectric helmet with flexible thermoelement regardless of the energy source (DC power supply or vehicle battery). In the invention, the temperature control unit is optimized according to the structure of the flexible thermoelectric helmet. It includes adapting the control parameters suitable for the cooling and heating control system to the power supply in line with the system concept. The fact that the power supply is microprocessor-based will also ensure that the user can program many relevant parameters in accordance with the purpose in question. The recording of all malfunctions related to the welding status will enable the necessary system information to be evaluated by the control circuit during use. In this case, the adaptation and optimization of the source to the various models to be made will be facilitated. The same control system ensured that the temperature of the flexible thermoelectric helmet was controlled proportionally (PID), keeping the electricity consumption of the device at a minimum. In addition, the control circuit has a structure that can operate the vehicle liquid cooler in cooling or heating mode or switch from one mode to another, which can operate from low currents to high currents and includes the switching elements such as transistor, thyristor, triac, and 4 relays and the current direction of which can be controlled digitally. A transistor structure was used to drive the relays. This structure was controlled with Arduino digital pins. When the relevant pin was logically made HIGH, the transistor was triggered and put into conduction, and current flow between collector and emitter was provided. In this case, the relay magnetized and changed the contact, and the normally open contact (no) and the common (c) terminal were connected. 4 digital pins were used to control 4 relays. There are many different types of microcontrollers on the market. Arduino programming was preferred because it is easy to program, open-code and economical. Arduino Nano model was used by being mounted on the control circuit with its compact feature. The temperature control system has a structure that can measure and control the inner surface temperature of the thermoelectric helmet or the temperature of the cooled liquid. The measured temperature is shown on the led display. LM35 semiconductor sensor was used for temperature measurement. The LM35 heat temperature sensor is simply a transistor. When we supply 5 volts from the collector (foot number 1) and connect the Emitter (foot number 3) to the ground, a current flows through the Base (foot number 2) and emitter. The current flowing through the base is at the level of mA and changes analogously with temperature. Since the sensor parameters change analog, we need to connect them to the analog pins of Arduino. It can measure between −55° C. and 150° C. The system works in 2 modes. In cooling and heating mode. Two push buttons were used for the selection of modes. When one of the buttons is pressed, the current applied polarity applied to the load is changed. While working in any mode, the temperature was measured every second and it was checked whether it reached the set value. If the set value has been reached, the relevant relays have been turned into the passive status, and the current applied to the load is interrupted. The invention has a special PID DC power and a smart measurement, recording, and control system that can measure and record the temperatures at various depths and the amount of heat released from the brain without interfering with the human brain, and converts them to 3D images without any harm to the patient. It is a portable and inexpensive new smart thermoelectric brain hypothermia thermohypotherm and thermography device.

Flexible combined hoses, which are a part of the smart thermoelectric brain hypothermia thermohypotherm and thermography device, have been calculated, designed, and produced based on the information and findings obtained scientific theoretical and experimental research. The electrical cables and hoses used here have been selected according to the medical device standards, the current to pass over it, and the fluid flow rate. Since the maximum current drawn by the thermoelectric helmet is DC40 A and the flow rate of water circulated from the copper pipes (heated side) of the flexible thermoelements forming the helmet is at least 120 Liter/hour, cables and silicon hoses have been chosen accordingly. The invention consists of a thermoelectric helmet (TEH) and a power control unit (PCU) equipped with intelligent software and special flexible combined hoses (9) with two sockets that provide the electricity and water connection between TEH and PCU, which tin connect (or separate) each other. It is a portable and cheap Smart Thermoelectric Brain Hypothermia Thermohypotherm and Thermography Device.

The special computer software, which makes the smart measurement, recording, and control system in the special power-control unit (PCU) of the Thermohypotherm and Thermography device intelligent, has been produced based on the information and findings obtained a result of basic scientific, theoretical and experimental research. Since the power supply or battery circuit used in the invention is microprocessor-based and the invention has a special PID power and temperature measurement and control circuit, special computer software has been prepared to ensure that these two systems are compatible with each other and that the thermoelectric helmet as a whole works with maximum efficiency. Arduino Nano programming language is used for this. In addition, there is an Arduino Nano development board on the unit. Thus, it can communicate with the computer using the USB port, and the software can be updated very easily. The invention is a new smart thermoelectric brain hypothermia thermohypotherm and thermography device equipped with special smart software.

Since the thermoelectric helmet is used directly, a cooling device can be turned into a heater machine by easily changing the direction of the current applied to the device. In addition, the device performs treatment and diagnosis procedures at the same time. The invention is a new smart thermoelectric brain hypothermia thermohypotherm and thermography device that combines treatment and diagnosis, cooling, and heating function in one device.

The description and operation of the invention can be explained as follows;

The smart thermoelectric brain hypothermia thermohypotherm and thermography device can be operated after the patient is attached or attached. When the helmet is operated without being attached to the patient's skull, the helmet's inner surface can be cooled down to the set temperature in a few minutes (generally, the temperature setting is not below −3° C. to prevent damage to the skull skin). When the application is made in this way, the cooling or heating time of the brain can be reduced to less than 5 minutes. To operate the device, a flexible thermoelectric helmet is attached to the patient's skull; first, the main program is selected on the touch panel according to the treatment or diagnosis that the doctor wants to do. If the application is for therapeutic purposes, the Thermohypotherm program is selected and how many degrees, how fast the patient's brain will be cooled (or heated), how many minutes will be kept at this temperature (depending on the case, this period may take days) and then to what degree, how fast it will be heated (or cooled). Data such as how many minutes will be kept at this temperature and when the system will be shut down automatically are entered into the system, and the device is started. In addition, the temperature values, time graphs, and distribution maps of the brain will be visually displayed on the screen of the device, either during or after hypothermia. If the application is for diagnostic purposes, the Thermography program is selected, and first of all, the temperatures of the patient at different points on the skull (zero depth of the brain) are measured, time graphs and distribution maps are recorded visually. Then, brain hypothermia is performed, and the values of temperatures of the brain at the desired depth, time, temperature graphs, and distribution maps are recorded visually and numerically. In addition, in this case, the device also performs the treatment. The obtained findings and graphics are automatically compared with the results obtained for various cases and stored in the device's memory, and the diagnosis of the disease is made in a few minutes.

The industrial applicability and application of the invention can be explained as follows.

The smart thermoelectric brain hypothermia thermohypotherm and thermography device is a device that can cool or heat very quickly because it is produced using the superior advantages of highly flexible thermoelectric technologies. On the other hand, since the use of this high technology ensures that the invention consists of simple parts and can be easily manufactured and assembled, the production cost of the device is at least 10 times less than the cost of the existing cooler and brain tomography devices. Accordingly, the invention is a new Smart thermoelectric brain hypothermia thermohypotherm and thermography device that is low in cost and easy to mass-produce.

The fact that the invention is light, silent, portable, and operable in vehicles will significantly increase the use of the device.

The invention is a new smart thermoelectric brain hypothermia thermohypotherm and thermography device that combines the function of harmless and uncomplicated treatment and diagnosis, cooling, and heating in one device, used for therapeutic or diagnostic purposes.

Smart electronic hardware and software will be developed with the development of technology, flexible thermoelement, and flexible thermoelectric helmet. With the shrinkage of basic parts such as the power supply or battery and closed liquid circulation unit, the smart thermoelectric brain hypothermia thermohypotherm and thermography device will turn into a personnel cooler that provides comfort with mildly controlled brain hypothermia, especially for healthy people. In this case, the number of production and sales of the invention will increase as much as the number of laptop computers or mobile phones, and since the export of the device will increase, a large amount of foreign currency input will be provided to the country.

1) A smart thermoelectric brain hypothermia thermohypotherm and thermography device comprising the following:
a thermoelectric flexible helmet,
a plurality of flexible thermoelements made of a soft, insulating, antibacterial material and placed in a plurality of slots on it,
a bottom plate,
a plurality of water collectors that provide fluid circulation through copper pipes of thermoelements, connect them to each other,
a plurality of flexible silicone hoses that allow hydraulic connection,
a flexible multi-copper wire that allows thermoelements to be electrically connected to each other,
a top sheet made of a flexible soft, insulating, antibacterial material like leather that covers all these parts,
a flexible filler filling the spaces between the thermoelements between the lower and upper sheets,
a flexible thermoelectric module helmet that can take the shape of a human skull,
a metal connector made as a hemisphere and fixed on the thermoelectric flexible helmet,
a flexible combo consisting of a plurality of plug-in electrical connection cables, a water pipe and a plurality of electrical wires metal fitting and combined hose fitting fixed to the end of the hose, a rough part of the fitting fixed to the helmet, the rough part of the fitting fixed to the combined hose end,
a closed water pump to remove the heat released from the heated surface of the thermoelements inside a casing,
a radiator-fan circulation system,
a DC SMPS power supply is placed and smart software equipped to make the helmet more efficient and economical,
a touch control panel,
an on-off button placed on the front panel,
an LED indicator,
a connection to a power control unit (PCU) consisting of four wheels, fixed on the rear panel of the case;
wherein the thermoelectric flexible helmet, the flexible combined hoses, and the power control unit are easily connected and separated from each other.

2) The smart thermoelectric brain hypothermia thermo-hypotherm and thermography device according to claim 1, wherein the flexible thermoelectric helmet-module is made of a single piece hemisphere that can take the shape of a human skull, The DC power it draws is 12V×40 A=480 W; it has a module with a cooling capacity of 240 W is specially made of a total of 120 flexible thermoelectric semiconductors (2), of which 120 N and 120 P-type thermoelectric semiconductors with a diameter of 8 mm and a length of 5 mm, The thermoelectric semiconductors produced as 1×N and 1×P-type cylindrical or flat parallelepipeds are produced by soldering a copper pipe with a special solder, which is produced in a way that will ensure the sealing of the silicone hoses (5) attached on it and facilitate the construction of helmets by processing a single piece of copper wire on one side and a single piece on the other.

3) The smart thermoelectric brain hypothermia thermo-hypotherm and thermography device according to claim 1, wherein the device is designed to cool the sick human brain with at least 120 W cooling capacity and 0.5° C./min cooling capacity in as little as 5 minutes without exposing the human to harmful effects such as radiation and magnetic field, it cools locally from the outside in a controlled manner with its speed, it can keep the brain's temperature at 28° C.-30° C. for the required time and ensure that the brain is heated to normal temperature at a controlled rate of 0.5° C./min after hypothermia, it treats brain hypothermia and at the same time combines the cooling and warming function of the patient under anesthesia in one device, it is a treatment device with the necessary hardware and software to treat patients by cooling the brain quickly and controlled up to 30° C. in brain diseases, anesthesia, cardiology, and other cases, especially Covid-19 patients exposed to severe pain, high fever, and hypoxia.

4. The smart thermoelectric brain hypothermia thermo-hypotherm and thermography device, according to claim 1, wherein the the smart device is configured to precisely measure, record, and display the temperatures and heat amounts in the brain (at different depths), with making use of the new method without interfering with the human brain, wherein the device also converts the data into a 3D image, wherein the device diagnoses all kinds of diseases without harming the patient and treats the patient by applying local hypothermia by directly contacting the skull without using any intermediates, wherein the device is portable and inexpensive, wherein the device has all the necessary hardware and software for a thermo-hypotherm and thermography device.

5. The smart thermoelectric brain hypothermia thermo-hypotherm and thermography device, according to claim 1, wherein the feature of the smart thermoelectric brain hypothermia thermohypotherm and thermography device: The device has a power supply of 12V×40 A=480 W, which is taken from the vehicle battery or the electrical network to perform a treatment and diagnosis, cooling and heating functions simultaneously, wherein, since the device is based on a microprocessor that can transfer electrical power without loss, it can be programmed by the user, and all faults related to the source status can be recorded, wherein, the control circuit can evaluate the necessary system information during use, wherein, the temperature control unit, which facilitates the adaptation and optimization to various models to be made, can control the temperature of the helmet proportionally (PID) and keeps the electricity consumption of TEH at a minimum level, has been optimized according to the structure of the flexible thermoelectric helmet, wherein, the device operates in the cooling or heating mode of the helmet, including the adaptation of the control parameters suitable for the cooling and heating control system to the power supply in line with the system concept, wherein, the device can switch from one mode to the other, from low currents to high currents, and can be digitally controlled for the current direction, including switching elements such as transistor, thyristor, triac, and 4 relays H-bridge diagram, wherein, this structure, which can drive relays with a transistor structure, is controlled by Arduino digital pins, wherein, the device measures and controls the internal surface temperature of the temperature control system thermoelectric cooler or the temperature of the cooled liquid with a semiconductor sensor such as LM35, and wherein, the device has a LED display and a PID DC power and temperature control unit that allows the helmet to operate in cooling and heating mode.

6. The smart thermoelectric brain hypothermia thermohypotherm and thermography device, according to claim 1, wherein the device is configured to diagnose all kinds of diseases, at the same time it cools the patient's brain in 5 minutes with a cooling capacity of at least 120 W and a rate of 0.5° C./min to treat hypothermia, wherein the device is configured to also keep the brain temperature at 28° C.-30° C. for the required time and wherein after hypothermia, flexible thermoelements (2) and flexible thermoelectric helmet can throw off the thermal power of at least 240 W, which is necessary to ensure that the brain is heated to normal temperature in a controlled manner and at a rate of 0.5° C./min., and wherein the device has a closed structure with a water pump, radiator-fan circulation system, which provides cooling of the heated surface (to keep it at room temperature) and has a flow rate of at least 120 L/min.

7. The smart thermoelectric brain hypothermia thermohypotherm and thermography device, according to claim 1, wherein the flexible combined hose (FCH) (9), which is a part of the system that makes up the device and can be easily connected and separated from each other, power, and control unit with TEH, a flexible thermoelectric helmet, wherein the device provides electrical connections, water connections, and information communication connections between (PCU) at the same time, wherein the device includes a structure that can pass electrical cables through the water hose, when necessary, carry at least DC40 A current, and provide at least 120 L/min water flow, electrical cables, and hoses selected according to medical device standards.

8. The smart thermoelectric brain hypothermia thermohypotherm and thermography device, according to claim 1, wherein the DC SMPS power and temperature control system is a microprocessor based PID controlled; the device ensures that these two systems are compatible with each other and the helmet as a whole works with maximum efficiency, wherein the helmet consists of 120 flexible thermoelectric thermoelements (2), when the helmet is worn on the patient, it not only provides the cooling or heating function at different points but also the measurement of the amount of heat released from these points and the desired temperatures in the brain depth, wherein the device includes computer-specific software written in Arduino and similar programming languages, enabling heat and temperature mapping of the brain with techniques such as 3D brain heat mapping and the device to perform diagnostic (thermography) and treatment with cooling (thermohypotherm) functions simultaneously.

9. The smart thermoelectric brain hypothermia thermohypotherm and thermography device, according to claim 1, wherein the device is configuring to make cooling and heating by easily changing the direction of the current applied to the flexible thermoelectric helmet that makes up the device, it measures the amount of heat released in at least 120 different points in the brain with new temperature and heat measurement methods and techniques, and wherein, by measuring the temperatures in the depth of the brain, the device makes a heat and temperature map of the brain and diagnoses the disease (thermography) and treats with cooling (thermo-hypotherm) in one device.

10) The smart thermoelectric brain hypothermia thermohypotherm and thermography device, according to claim 5 wherein the DC SMPS power and temperature control system is a microprocessor based PID controlled; the device ensures that these two systems are compatible with each other and the helmet as a whole works with maximum efficiency, wherein the helmet consists of 120 flexible thermoelectric thermoelements (2), when the helmet is worn on the patient, it not only provides the cooling or heating function at different points but also the measurement of the amount of heat released from these points and the desired temperatures in the brain depth, wherein the device includes computer-specific software written in Artificial Intelligence, Arduino, and similar programming languages, enabling heat and temperature mapping of the brain with techniques such as 3D brain heat mapping and the device to perform diagnostic (thermography) and treatment with cooling (thermohypotherm) functions simultaneously.
